(19) United States
(12) Patent Application Publication
(10) Pub. No.: US 2016/0008189 A1
(43) Pub. Date: Jan. 14, 2016

(54) DISPOSABLE DIAPER WITH POCKET CONTAINING ONE OR MORE WIPES

(71) Applicants: LOUIS RONALD EZZO, RIVERTON, IL (US); ELSA BERNICE EZZO, RIVERTON, IL (US)

(72) Inventors: LOUIS RONALD EZZO, RIVERTON, IL (US); ELSA BERNICE EZZO, RIVERTON, IL (US)

(21) Appl. No.: 14/798,874

(22) Filed: Jul. 14, 2015

Related U.S. Application Data
(60) Provisional application No. 61/998,951, filed on Jul. 14, 2014.
(60) Provisional application No. 61/996,989, filed on Nov. 5, 2014.

(51) Int. Cl. A61F 13/84; A61F 13/15; A61F 13/505

(52) U.S. Cl. CPC A61F 13/84; A61F 13/505; A61F 13/15699; A61F 2013/8476; A61F 2013/15048

(57) ABSTRACT

The invention provides a disposable diaper with a pocket containing one or more sanitary wipes. The pocket is constructed on the outer layer of the diaper and provides a closed, moisture-impermeable chamber within which one or more moistened wipes are stored. The pocket can include perforations, perforation strips or a tear strip that allows the user to open the pocket and access the one or more wipes by tearing.

### CROSS-REFERENCE TO RELATED APPLICATIONS

This application claims priority to U.S. provisional patent application No. 61/998,951, filed Jul. 14, 2014, and U.S. provisional application No. 61/996,989, filed Nov. 5, 2014, the contents of which are incorporated herein by reference in their entirety.

### BACKGROUND

Diapers are useful for infants, children who are not trained to use a toilet, those with incontinence or other physical or mental disability, as well as individuals engaging in activities in which access to a toilet is limited or impractical such as astronauts and divers. Diapers can also be used for various animals such as pets that have not been housebroken or are older, sick or have become incontinent.

### SUMMARY OF THE INVENTION

The invention provides an improved disposable diaper with a pocket containing one or more sanitary wipes. The pocket is constructed on the outer layer of the diaper and provides a closed, moisture-impermeable chamber within which one or more moistened wipes are stored. A diaper of the invention allows the user convenient and easy access to the one or more moist wipes as needed.

In one aspect, the invention provides a disposable diaper having one or more integrated wipes contained within a moisture-impermeable pocket integral with the diaper outer layer, the one or more wipes being accessible by tearing through the diaper outer layer.

In some embodiments of a diaper of the invention, the moisture-impermeable pocket includes an inner panel bonded along its entire peripheral edge to the reverse side of the diaper outer layer so as to form a closed pocket between the pocket inner panel and the portion of the diaper outer layer that is surrounded by the bonded peripheral edge, and the pocket inner panel and the portion of the diaper outer layer that is surrounded by the bonded peripheral edge includes a moisture-impermeable material so as to render the pocket moisture-impermeable. In some embodiments, the moisture-impermeable material includes a plastic liner or coating disposed on opposing surfaces of the diaper outer layer and pocket inner panel that form the pocket. In some embodiments, the moisture-impermeable material includes nylon, polyester, polyethylene, polypropylene, or any combination thereof.

In some embodiments, the pocket inner panel is bonded along its entire peripheral edge to the diaper outer layer using an adhesive. In some embodiments, the pocket inner panel is heat bonded or chemically bonded along its entire peripheral edge to the diaper outer layer.

In some embodiments of a diaper of the invention, the diaper outer layer includes perforations that enable the user to access the one or more wipes stored within the moisture-impermeable pocket by tearing about the perforations. In some embodiments, the diaper outer layer includes a perforated strip that enables the user to access the one or more wipes stored within the moisture-impermeable pocket by tearing along the perforated strip. In some embodiments, the moisture-impermeable pocket includes a tear tab or tear strip that enables the user to access the one or more wipes stored within the moisture-impermeable pocket by tearing from the tab or along the strip.

In some embodiments of a diaper of the invention, the moisture-impermeable pocket includes an outer panel bonded along its entire peripheral edge to the obverse surface of the diaper outer layer so as to form a closed pocket with the portion of the diaper outer layer that is surrounded by the bonded peripheral edge, and the pocket outer panel and at least the portion of the diaper outer layer that is surrounded by the bonded peripheral edge includes a moisture-impermeable material so as to render the closed pocket moisture-impermeable. In some embodiments, the pocket outer panel has the same material as the diaper outer layer. In some embodiments, the moisture-impermeable material includes a plastic liner or coating disposed on opposing surfaces of the pocket outer panel and diaper outer layer that form the pocket. In some embodiments, the moisture-impermeable material includes nylon, polyester, polyethylene, polypropylene, or any combination thereof.

In some embodiments, the pocket outer panel is bonded along its entire peripheral edge to the diaper outer layer using an adhesive. In some embodiments, the pocket outer panel is heat bonded or chemically bonded along its entire peripheral edge to the pocket outer layer.

In some embodiments of a diaper of the invention, the pocket outer panel includes perforations that enable the user to access the one or more wipes stored within the moisture-impermeable pocket by tearing about the perforations. In some embodiments, the pocket outer panel includes a perforated strip that enables the user to access the one or more wipes stored within the moisture-impermeable pocket by tearing along the perforated strip. In some embodiments, the pocket outer panel includes a tear tab or tear strip that enables the user to access the one or more wipes stored within the moisture-impermeable pocket by tearing from the tab or along the strip.

In another aspect, the invention provides a method for producing a disposable diaper that involves: (a) placing one or more wipes between the reverse side of a diaper outer layer and a pocket inner panel, and (b) bonding the peripheral edge of the pocket inner panel to the reverse side of the diaper outer layer to form a closed pocket containing the one or more wipes disposed between the reverse side of the diaper outer layer and the pocket inner panel, the pocket inner panel and at least the portion of the diaper outer layer that is surrounded by the bonded peripheral edge having a moisture-impermeable material so as to render the pocket moisture-impermeable.

In another aspect, the invention provides a method for producing a disposable diaper that involves: (a) placing one or more wipes between a pocket outer panel and the obverse surface of a diaper outer layer, and (b) bonding the peripheral edge of the pocket outer panel to the obverse surface of the diaper outer layer to form a closed pocket containing one or more wipes disposed between the pocket outer panel and the portion of the diaper outer layer surrounded by the bonded peripheral edge, the pocket outer panel and at least the portion of the diaper outer layer that is surrounded by the bonded peripheral edge having a moisture-impermeable material so as to render the closed pocket moisture-impermeable.

Any feature or combination of features described herein are included within the scope of the present invention provided that the features included in any such combination are not mutually inconsistent as will be apparent from the context, this specification and the knowledge of one of ordinary skill in the art.

Unless otherwise defined, all technical and scientific terms used herein have the same meaning as commonly understood by one of ordinary skill in the art to which this invention belongs. In case of conflict, the present specification, including definitions, will control. In addition, the materials, methods, and examples are illustrative only and not intended to be limiting. Although methods and materials similar or equivalent to those described herein can be used to practice the invention, suitable methods and materials are described below.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

Other features and advantages of the invention will be apparent from the following detailed description and from the claims.

### DESCRIPTION OF THE FIGURES

FIGS. 1A-1B are diagrams of diaper 10 with pocket 16 containing one or more wipes, diaper 10 being in a folded and closed configuration (1A) and in an unfolded open configuration (1B).

FIG. 2 is an exploded diagram illustrating the structure of diaper 10 in the unfolded open configuration as viewed from a top perspective.

FIGS. 3A-3C are diagrams of the outer layer of diaper 10 illustrating the structure and location of pocket 16 including an exploded view of the structure of pocket 16 (3A), an enlarged, exterior perspective view of pocket 16 in an unopen configuration (3B), an enlarged, exterior perspective view of pocket 16 in an opened configuration (3C), and a cross-sectional view along lines 3D-3D (3D).

FIGS. 4A-4B are diagrams of diaper 100 with pocket 160 containing one or more wipes including a diagram of diaper 100 in a folded and closed configuration as viewed from the front (4A), and an exploded diagram illustrating the structure of the outer layer of diaper 100 with pocket 160 as viewed from an exterior perspective (4B).

FIGS. 5A-5C are diagrams of pocket 160 including an enlarged perspective view of pocket 16 in the closed configuration (5A), an enlarged perspective view of pocket 16 in an opened configuration (5B), and a cross-sectional view along line 5C-5C (5C).

FIG. 6 is an enlarged diagram of an embodiment of a diaper of the invention in which the pocket includes a perforated strip to facilitate opening the pocket containing one or more wipes.

FIG. 7 is a diagram of an embodiment of a diaper of the invention in the unfolded open configuration as viewed from its exterior side illustrating two possible locations of the pocket containing one or more wipes.

### DETAILED DESCRIPTION OF THE INVENTION

The invention provides an improved disposable diaper with a pocket containing one or more sanitary wipes. The pocket is constructed on the outer layer of the diaper and provides a closed, moisture-impermeable chamber within which one or more moist wipes are stored thereby allowing the user convenient and easy access to the one or more moist wipes as needed. Specific embodiments of the invention are described in the following examples, which do not limit the scope of the invention described in the claims.

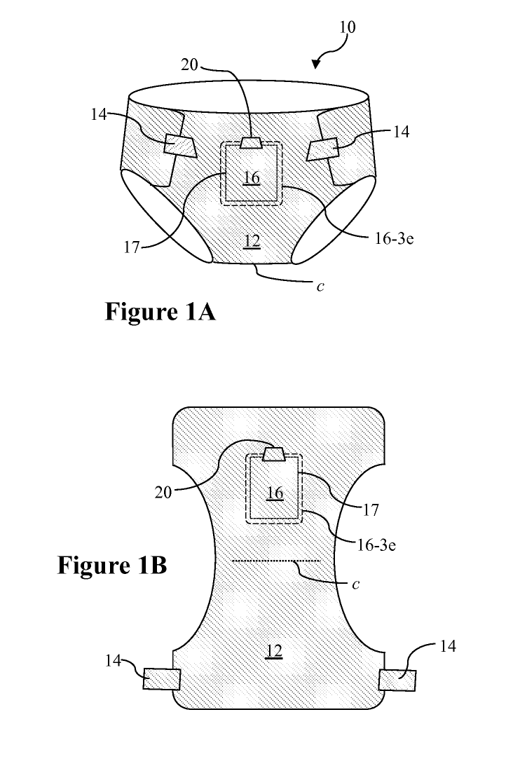

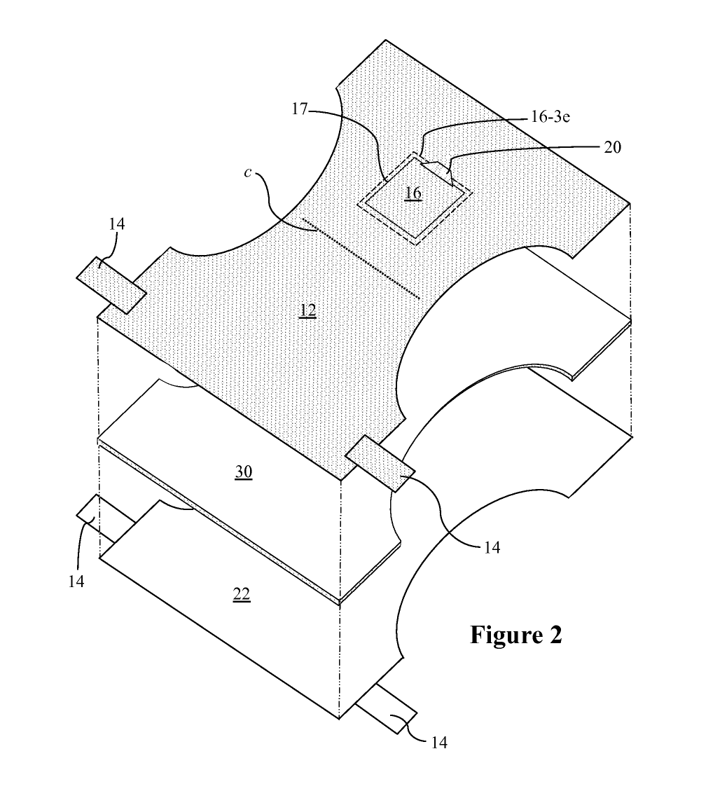

FIGS. 1A-1B, 2, and 3A-3D illustrate an embodiment of a diaper of the invention. FIG. 1A illustrates diaper 10 folded and secured in a closed configuration. FIG. 1B illustrates diaper 10 in an unfolded, open configuration. Diaper 10 includes pocket 16, which contains one or more sanitary wipes. Pocket 16 is constructed with perforation 17 to facilitate opening of the pocket for access to the sanitary wipes. Tab 20 is optional and facilitates grip and opening of pocket 16. Diaper 10 is constructed with outer layer 12, inner layer 22, and absorbent layer 30 disposed between outer layer 12 and inner layer 22 as shown in FIG. 2.

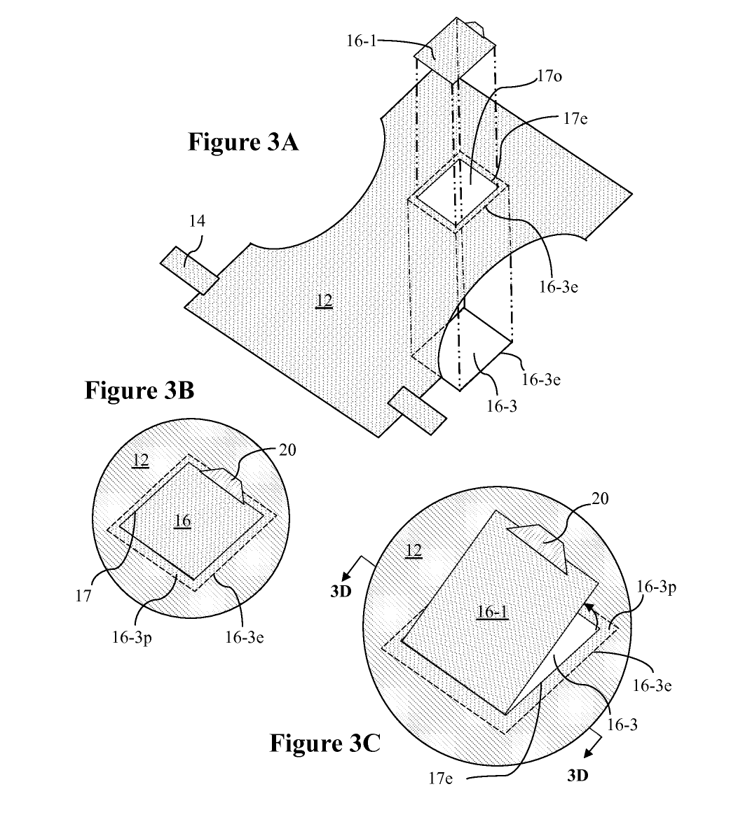

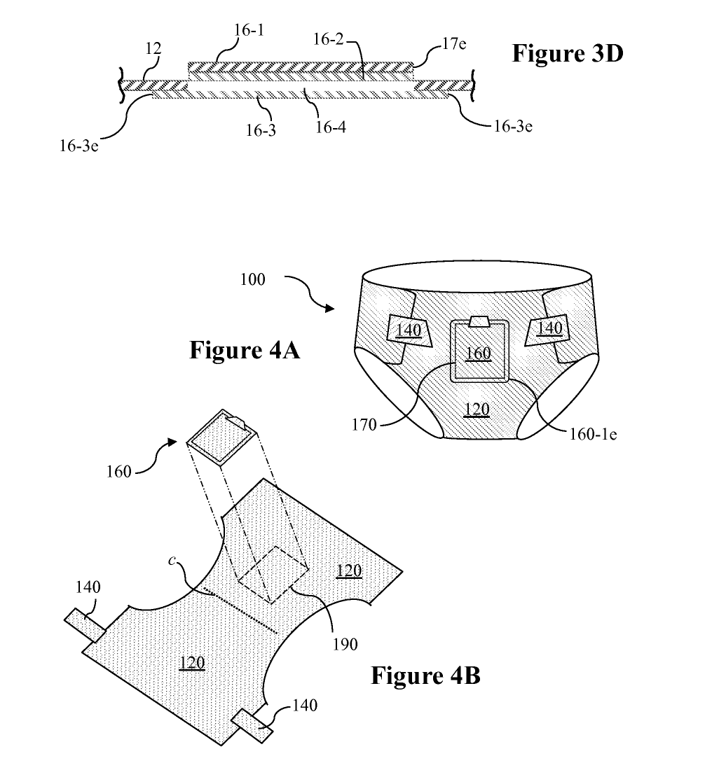

Pocket 16 is integrated within diaper outer layer 12 as illustrated in FIGS. 3A-3D. In this embodiment, pocket 16 is constructed as part of diaper outer layer 12. More specifically, diaper outer layer 12 includes perforation 17 (FIGS. 1A-1B and FIG. 2). Tearing along perforation 17 on diaper outer layer 12 generates pocket outer panel 16-1, opening 17o (a cut-out in diaper outer layer 12), and edge 17e as shown in FIG. 3A. Lifting pocket outer panel 16-1 off the outer surface of the diaper as indicated by the arrow in FIG. 3C opens pocket 16 allowing the user to access the one or more wipes stored in the chamber between pocket outer panel 16-1 and pocket inner panel 16-3 (FIG. 3C). Thus, pocket 16 includes: (a) the portion of diaper outer layer 12 that corresponds to pocket outer panel 16-1 generated upon tearing along perforation 17, and (b) pocket inner panel 16-3 that is visible when pocket outer panel 16-1 is pealed from the body of diaper 10 (FIG. 3C).

Pocket inner panel 16-3 is configured wider and longer than pocket outer panel 16-1 as indicated by the relative location of edge 16-3e with respect to edge 17e (FIGS. 3A-3D). The length and width of pocket outer panel 16-1 extends to perforations 17, while the length and width of pocket inner panel 16-3 extends beyond perforations 17 to edge 16-3e as illustrated in FIGS. 3A-3D. Peripheral edge portion of pocket inner panel 16-3 extending beyond perforation 17, i.e. portion 16-3p, is secured to the reverse side of diaper outer layer 12 by adhesive, heat or chemical bonding as shown in FIG. 3D. Pocket 16 also includes moisture-impermeable liner 16-2 secured to the reverse side of pocket outer panel 16-1 as shown in the cross-sectional view of FIG. 3D in which pocket outer panel 16-1 is shown partially lifted off the surface of diaper 10.

Thus, pocket chamber 16-4 where one or more sanitary wipes are stored is disposed between (a) pocket outer panel 16-1 to which moisture-impermeable liner 16-2 is secured, and (b) pocket inner panel 16-3, which is secured along its peripheral edge portion to the reverse side of diaper outer layer 12 to close the opening in diaper outer layer 12 when pocket outer panel 16-1 is formed upon tearing along perforation 17. As such, pocket inner panel 16-3 is disposed between the diaper outer layer 12 and absorbent layer 30. Peripheral edge portion 16-3p of pocket inner panel 16-3 can be secured to the reverse surface of diaper outer layer 12 using any means known to those of skilled in the art including stitching, bonding with an adhesive or through thermal or chemical bonding.

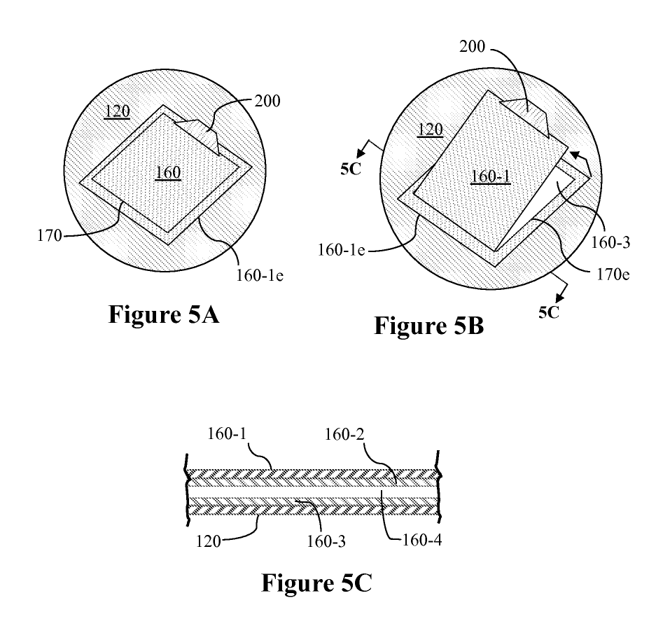

Another embodiment of the invention is illustrated in FIGS. 4A-4B and FIGS. 5A-5C. In diaper 100, pocket 160 is secured directly to the surface of exterior layer 120 as shown in FIG. 4A-4B. In this embodiment, the reverse side of pocket 160 is secured to the obverse side of diaper outer layer 120 at position 190. FIGS. 5A-5C illustrate the structure of pocket 160, which can be opened by tearing, for example, along perforation 170. Tab 200 provides a grip for tearing open pocket 160 and lifting pocket outer panel 160-1 as shown in FIG. 5B. Tab 200 is optional, as pocket 160 can be opened by tearing in any manner that allows the user access to the sanitary wipes stored within pocket 160. Thus, pocket 160 is constructed with an outer panel 160-1 and inner panel 160-3. Liner 160-2 is attached to the reverse side of pocket outer layer 160-1, while pocket inner panel 160-3 is attached directly to diaper outer layer 120 as shown in the cross-sectional view of FIG. 5C. In some embodiments, diaper outer panel 160-1 can be of the same material as diaper outer layer 120, and liner 160-2 can be constructed of the same moisture-impermeable material as pocket inner panel 160-3 to form the moisture-impermeable chamber 160-4 within which sanitary wipes are stored. As constructed, pocket 160 is directly secured to diaper outer layer 120 using any means known to those of skilled in the art for bonding of pocket inner panel 160-3 to diaper outer layer 120 including bonding with an adhesive or by thermal or chemical bonding.

In general, the one or more sanitary wipes incorporated within a diaper of the invention can be moistened or dry, flushable or unflushable, and scented or unscented. As used herein the term “sanitary wipes” refers to any cleansing cloths or wipes used for cleansing or hygiene purposes. Sanitary wipes include cleansing cloths used on a person such as an infant, child or adult, or on an animal, for cleansing or disinfecting purposes. The wipes can include one or more ingredients known to those of skill in the art or typically included in sanitary wipes. Non-limiting examples include one or more antibacterial agents, cleansing agents, preservatives, vitamins, moisturizer, other skin care ingredients, therapeutic agents, or any combination thereof.

The pocket on the diaper within with the sanitary wipes are stored can be of any shape or size effective to contain a select number of wipes, for example, 1, 2, 3, 4, 5, 6 or more wipes. For example, the pocket can have dimensions of about 2.5 inches×about 3.5 inches. The pocket can be constructed with perforations for ease of access to the wipes. The perforations can be in any configuration or pattern to facilitate tearing of the pocket outer panel for access to the wipes. The perforations can be in the form of a rectangular or square box as illustrated in FIGS. 1-5. Alternatively, the performations can be in one or more vertical, horizontal or diagonal lines such as a cross or perforated strip on the surface of the pocket to allow the user to tear open the pocket outer panel to access the wipes stored within. In general, perforations are optional, and where included, any perforation pattern can be employed so long as it enables the user to tear open the pocket and access the sanitary wipes contained within.

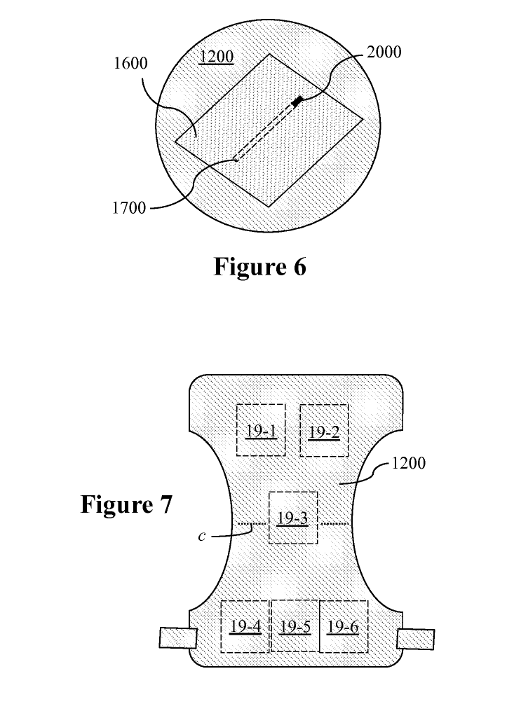

The pocket can, optionally, include means for tearing open the pocket such as a tab or strip of material or tape mounted to the obverse or reverse side of the pocket outer panel so long as a portion of the tear means is accessible on the outside of the pocket or diaper to facilitate opening the pocket. An example of a tear means include tab 20 secured to one portion of the pocket as illustrated in FIGS. 1-5 is an example. Alternatively, the tear means can be a perforated strip, or other strip, tape, string or thread secured on the obverse or reverse surface of the pocket outer panel to enable the user to tear open the pocket by pulling on the string, tape or thread. An example of a means for tearing open the pocket include, for example, tear strip 2000 illustrated in FIG. 6. Where a tear means is included, the tear means can be placed at any location that facilitates opening the pocket for access to the wipes stored within.

The pocket can be constructed of any one or more materials known to those of skilled in the art that is conducive to tearing and/or is moisture-impermeable such as paper and plastic. The pocket outer panel can be constructed of a moisture-impermeable material or a moisture-permeable material that is lined, coated or otherwise treated with a moisture-impermeable material. Where the pocket outer panel is constructed with a moisture-permeable material, the reverse side can be lined, coated or otherwise treated with a moisture-impermeable material. Similarly, the pocket inner panel can be constructed of moisture-permeable material or a moisture-impermeable material. Where it is constructed with a moisture permeable material, its obverse side can be similarly lined, coated or otherwise treated with a moisture-impermeable material. The moisture-impermeable material on the pocket outer panel and pocket inner panel can be the same or different materials.

A diaper of the invention can include a pocket having an outer panel composed of the same material as the diaper outer layer and a pocket inner panel that is composed of a moisture-impermeable material bonded along its peripheral edge portion to the reverse side of the diaper outer layer so as to form a moisture impermeable chamber for holding one or more wipes. In this embodiment, the pocket inner panel is disposed between the diaper outer layer and the diaper absorbent layer.

A diaper of the invention can include a pocket having a moisture-impermeable outer panel and a moisture-impermeable inner panel, their peripheral edge portion bonded together to form a inner chamber containing one or more wipes. In this embodiment, the pocket can be bonded to the diaper outer layer on its obverse or reverse side.

The moisture impermeable material can be any synthetic or nonsynthetic plastic, bioplastic or petroleum-based plastic, or plastic-treated material known to those of skilled in the art. Non-limiting examples of moisture impermeable materials that can be used to construct the pocket in a diaper of the invention include polyethylene, polylactic acid, rubber, polyvinyl chloride, polyurethane, silicone elastomer, fluoropolymers, wax or any combination thereof.

Materials that can be used to construct the pocket including its outer panel and/or inner panels are known to those of skill in the art and can be any synthetic or nonsynthetic plastics and plastic resins. Non-limiting examples of materials that can be used to construct the pocket in a diaper of the invention include nylon, polyester, polyethylene (PE), polypropylene (PP) or cast polypropylene (CPP), polyurethane laminate, thermoplastic polyurethane, polyethylene terephthalate (PET), polyethylene terephthalate polyester (PETP), ortho-phthalaldehyde (OPA), ortho-phenylphenol (OPP), polyvinyl chloride (PVC), polyvinylidene chloride (PVDC), ethylene vinyl alcohol (EVOH), polyvinyl alcohol (PVAL), silicon oxides (SIOX), aluminum oxide (ALOX), polystyrene (PS), or any combination thereof. For example, a pocket of the invention can be composed of two outer polyester panels lined, coated or laminated with a polyethylene liner such as those used in flow wraps known to those of skilled in the art. A pocket of the invention can also be constructed of panels composed of a combination of polyethylene terephthalate, polyester and aluminum (AL). Other combinations of materials that can be used in constructed a pocket of the invention include: PET/ALU/PE; PET/ALU/PET/PE; PET/ALU/CPP; PETP/PE; PETPmet/PE; OPA/PE; OPP/OPP; mono foils for example, OPP, cellophane, aluminium, PVC, PET and paper; aluminium bands; paper/aluminium/PE or Surlyn; paper/PVDC/PE; paper/aluminium/PAN (Barex); laminates with barrier coatings based on EVOH, PVAL, SIOX, ALOX for solid sealing or peelable sealing; and laminates with hot seal laquers sealable against PE, PP, PET, PVC, and PS.

Components forming the pocket can be secured one to the other or to the the diaper outer panel using any means known to those of skilled in the art including by stitching, using an adhesive or by thermal or chemical bonding.

In a diaper of the invention, the pocket can be at any convenient location on the diaper, for example, on the exterior of the diaper at the front, near the crotch area or at the rear as indicated by boxes 19-1, 19-2, 19-3, 19-4, 19-5, and 19-6 illustrated in FIG. 7. Generally, the pocket is disposed at a location that does not typically become soiled, e.g. on the diaper outer layer, and is conveniently accessible, for example, at the front, crotch area or rear. A diaper of the invention can include any number of pockets, for example, one or two pockets as needed.

A diaper of the invention can be constructed in any size or shape as needed to accommodate any individual, for example, infant, toddler, child, or adult person. A diaper of the invention also can be configured to fit a select animal including, for example, a dog, cat, or horse. As used herein, the term “diaper” refers to any disposable garment configured for use, for example, as underpants, under circumstances in which access to a toilet is limited or a toilet is not available, or configured for use by one not toilet trained or having one or more illnesses or conditions such as, for example, incontinence, mobility impairment, severe diarrhea or dementia.

### OTHER EMBODIMENTS OF THE INVENTION

While the invention has been described in conjunction with the detailed description, the foregoing description is intended to illustrate and not limit the scope of the invention, which is defined by the scope of the claims. Other aspects, advantages, and modifications are within the scope of the following claims.

The terms and expressions that have been employed are used as terms of description and not of limitation, and there is no intent in the use of such terms and expressions to exclude any equivalent of the features shown and described or portions thereof. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. In addition, the invention has been described broadly and generically herein. Each of the narrower species and subgeneric groupings falling within the generic disclosure also form part of the invention.

As used herein and in the appended claims, the singular forms “a,” “an,” and “the” include plural reference unless the context clearly dictates otherwise. Under no circumstances may the patent application be interpreted to be limited to the specific examples or embodiments or methods specifically disclosed herein.

What is claimed is:

1. A disposable diaper comprising one or more integrated wipes contained within a moisture impermeable pocket integral with the diaper outer layer, the one or more wipes being accessible by tearing through the diaper outer layer.

2. The disposable diaper of claim 1, wherein the moisture-impermeable pocket comprises an inner panel bonded along its entire peripheral edge to the reverse side of the diaper outer layer so as to form a closed pocket between the pocket inner panel and the portion of the diaper outer layer that is surrounded by the bonded peripheral edge, and wherein the pocket inner panel and the portion of the diaper outer layer that is surrounded by the bonded peripheral edge comprise a moisture-impermeable material so as to render the pocket moisture-impermeable.

3. The disposable diaper of claim 2, wherein the moisture-impermeable material comprises a plastic liner or coating disposed on opposing surfaces of the diaper outer layer and pocket inner panel that form the pocket.

4. The disposable diaper of claim 2, wherein the moisture-impermeable material comprises nylon, polyester, polyethylene, polypropylene, or any combination thereof.

5. The disposable diaper of claim 2, wherein the pocket inner panel is bonded along its entire peripheral edge to the diaper outer layer using an adhesive.

6. The disposable diaper of claim 2, wherein the pocket inner panel is heat bonded or chemically bonded along its entire peripheral edge to the diaper outer layer.

7. The disposable diaper of claim 2, wherein the diaper outer layer comprises perforations that enable the user to access the one or more wipes stored within the moisture-impermeable pocket by tearing about the perforations.

8. The disposable diaper of claim 7, wherein the diaper outer layer comprises a perforated strip that enables the user to access the one or more wipes stored within the moisture-impermeable pocket by tearing along the perforated strip.

9. The disposable diaper of claim 2, wherein the moisture-impermeable pocket comprises a tear tab or tear strip that enables the user to access the one or more wipes stored within the moisture-impermeable pocket by tearing from the tab or along the strip.

10. The disposable diaper of claim 1, wherein the moisture-impermeable pocket comprises an outer panel bonded along its entire peripheral edge to the obverse surface of the diaper outer layer so as to form a closed pocket with the portion of the diaper outer layer that is surrounded by the bonded peripheral edge, and wherein the pocket outer panel and at least the portion of the diaper outer layer that is surrounded by the bonded peripheral edge comprise a moisture-impermeable material so as to render the closed pocket moisture-impermeable.

11. The disposable diaper of claim 10, wherein the pocket outer panel comprises the same material as the diaper outer layer.

12. The disposable diaper of claim 10, wherein the moisture-impermeable material comprises a plastic liner or coating disposed on opposing surfaces of the pocket outer panel and diaper outer layer that form the pocket.

13. The disposable diaper of claim 10, wherein the moisture-impermeable material comprises nylon, polyester, polyethylene, polypropylene, or any combination thereof.

14. The disposable diaper of claim 10, wherein the pocket outer panel is bonded along its entire peripheral edge to the diaper outer layer using an adhesive.

15. The disposable diaper of claim 10, wherein the pocket outer panel is heat bonded or chemically bonded along its entire peripheral edge to the pocket outer layer.

16. The disposable diaper of claim 10, wherein the pocket outer panel comprises perforations that enable the user to access the one or more wipes stored within the moisture-impermeable pocket by tearing about the perforations.

17. The disposable diaper of claim 16, wherein the pocket outer panel comprises a perforated strip that enables the user to access the one or more wipes stored within the moisture-impermeable pocket by tearing along the perforated strip.

18. The disposable diaper of claim 10, wherein the pocket outer panel comprises a tear tab or tear strip that enables the user to access the one or more wipes stored within the moisture-impermeable pocket by tearing from the tab or along the strip.

19. A method for producing the disposable diaper of claim 2 comprising (a) placing one or more wipes between the reverse side of a diaper outer layer and a pocket inner panel, and (b) bonding the peripheral edge of the pocket inner panel to the reverse side of the diaper outer layer to form a closed pocket containing the one or more wipes disposed between the reverse side of the diaper outer layer and the pocket inner panel, and wherein the pocket inner panel and at least the portion of the diaper outer layer that is surrounded by the bonded peripheral edge comprise a moisture-impermeable material so as to render the pocket moisture-impermeable.

20. A method for producing the disposable diaper of claim 10 comprising (a) placing one or more wipes between a pocket outer panel and the obverse surface of a diaper outer layer, and (b) bonding the peripheral edge of a pocket outer panel to the obverse surface of the diaper outer layer to form a closed pocket containing one or more wipes disposed between the pocket outer panel and the portion of the diaper outer layer surrounded by the bonded peripheral edge, and wherein the pocket outer panel and at least the portion of the diaper outer layer that is surrounded by the bonded peripheral edge comprise a moisture-impermeable material so as to render the closed pocket moisture-impermeable.
